(19) United States
(12) Patent Application Publication
(10) Pub. No.: US 2025/0381331 A1
(43) Pub. Date: Dec. 18, 2025

(54) SYSTEMS FOR OXYGENATOR

(71) Applicant: CDX Medical Technologies, LLC, New York, NY (US)

(72) Inventor: Gopal K. CHOPRA, New York, NY (US)

(73) Assignee: CDX Medical Technologies, LLC, New York, NY (US)

(21) Appl. No.: 19/240,716

(22) Filed: Jun. 17, 2025

Related U.S. Application Data
(60) Provisional application No. 63/661,380, filed on Jun. 18, 2024.

(51) Int. Cl. A61M 1/16

(52) U.S. Cl. CPC A61M 1/1698; A61M 1/1621

(57) ABSTRACT

An oxygenator system includes: a canister; a membrane in the canister to provide a gas transfer with blood; a blood inflow tube to deliver blood to the canister; and a blood outflow tube to deliver blood from the canister, wherein one or more of the blood inflow tube or the blood outflow tube includes a curved portion.

### CROSS-REFERENCE TO RELATED APPLICATION(S)

This application claims the benefit of priority to U.S. Provisional Patent Application No. 63/661,380, filed Jun. 18, 2024, the entirety of which is incorporated by reference herein.

### TECHNICAL FIELD

Various embodiments of the present disclosure relate generally to systems for an oxygenator.

### BACKGROUND

An oxygenator is a medical device that performs a similar function as a lung, and is designed to expose blood to oxygen, and, in some oxygenators, remove carbon dioxide from the blood. Some oxygenators (e.g., extracorporeal membrane oxygenation) include a membrane permeable to gas but impermeable to blood. Blood flows on the outside surface of the membrane, while oxygen and medical air flows inside the membrane, which permits the blood cells to absorb oxygen molecules directly. However, some oxygenators impart mechanical fluid forces to the blood, which may lead to adverse outcomes, such as thrombosis and bleeding. Some oxygenators have designs that reduce efficiency of blood flow and oxygen exchange, or have reduced thermal efficiency.

The present disclosure is directed to overcoming one or more of these above-referenced, or other, challenges.

### SUMMARY OF THE DISCLOSURE

In some aspects, the techniques described herein relate to an oxygenator system including: a canister; a membrane in the canister to provide a gas transfer with blood; a blood inflow tube to deliver blood to the canister; and a blood outflow tube to deliver blood from the canister, wherein one or more of the blood inflow tube or the blood outflow tube includes a curved portion.

In some aspects, the techniques described herein relate to an oxygenator system, wherein one or more of the blood inflow tube or the blood outflow tube includes a blood flow modifier.

In some aspects, the techniques described herein relate to an oxygenator system, wherein the blood flow modifier includes fins extending towards a central axis of the one or more of the blood inflow tube or the blood outflow tube.

In some aspects, the techniques described herein relate to an oxygenator system, wherein the blood flow modifier includes a straight portion relative to a longitudinal direction of flow.

In some aspects, the techniques described herein relate to an oxygenator system, wherein the blood flow modifier includes an angled or pitched portion relative to a longitudinal direction of flow.

In some aspects, the techniques described herein relate to an oxygenator system, wherein the curved portion is helical.

In some aspects, the techniques described herein relate to an oxygenator system, wherein one or more of the blood inflow tube or the blood outflow tube includes a funnel portion.

In some aspects, the techniques described herein relate to an oxygenator system, further including: a dissipating surface between the membrane and one or more of the blood inflow tube or the blood outflow tube.

In some aspects, the techniques described herein relate to an oxygenator system, wherein the dissipating surface further includes a separator including angled blades.

In some aspects, the techniques described herein relate to an oxygenator system, further including: a dissipating surface between the membrane and one or more of the blood inflow tube or the blood outflow tube.

In some aspects, the techniques described herein relate to an oxygenator system including: a first canister; and a connector to connect the first canister to a second canister.

In some aspects, the techniques described herein relate to an oxygenator system, wherein: the first canister includes a first blood flow inlet and a first blood flow outlet, and the second canister includes a second blood flow inlet to connect to the first blood flow outlet, and a second blood flow outlet.

In some aspects, the techniques described herein relate to an oxygenator system, wherein: the first canister includes a first gas inlet and a first gas outlet, and the second canister includes a second gas inlet to connect to the first gas outlet, and a second gas outlet.

In some aspects, the techniques described herein relate to an oxygenator system, wherein the first canister includes a first stacking feature, and the second canister includes a second stacking feature to connect to the first stacking feature to stack the second canister on the first canister.

In some aspects, the techniques described herein relate to an oxygenator system, wherein: the first canister is configured for a first flow rate, the second canister is configured for a second flow rate different from the first flow rate, and the first flow rate and the second flow rate include one or more of a gas flow rate or a blood flow rate.

In some aspects, the techniques described herein relate to an oxygenator system, wherein: the first canister is configured to transfer a first gas into blood, and the second canister is configured to remove a second gas from blood.

In some aspects, the techniques described herein relate to a membrane for an oxygenator system, the membrane including: one or more hollow fibers that are each wound in a helical shape, wherein the one or more hollow fibers are configured to exchange a gas inside the one or more hollow fibers with a fluid outside the one or more hollow fibers.

In some aspects, the techniques described herein relate to a membrane, wherein the membrane includes one or more spacers to provide an additional function to the membrane.

In some aspects, the techniques described herein relate to a membrane, wherein the one or more hollow fibers include inner helical pillars folded into one or more sheets and an outer helix coiled around the one or more sheets.

In some aspects, the techniques described herein relate to a membrane, wherein the helical shape includes double helix shapes.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 is an illustration of an oxygenator system, according to aspects of this disclosure.

FIG. 2A is an illustration of a hollow fiber for a membrane for an oxygenator system, according to aspects of this disclosure.

FIG. 2B is an illustration of the hollow fiber of FIG. 2A formed as a helical winding, according to aspects of this disclosure.

FIG. 2C is an illustration of multiple hollow fibers formed as multiple helical windings, according to aspects of this disclosure.

FIG. 2D is an illustration of multiple hollow fibers that are folded, according to aspects of this disclosure.

FIG. 3 is an illustration of a membrane of an oxygenator system, according to aspects of this disclosure.

FIG. 4 is an illustration of a membrane with inner and outer fibers, according to aspects of this disclosure.

FIG. 5 is an illustration of blood flow modifiers, according to aspects of this disclosure.

FIG. 6A is an illustration of a straight blood flow tube, according to aspects of this disclosure.

FIG. 6B is an illustration of blood flow tubes in a stacked helix configuration, according to aspects of this disclosure.

FIG. 6C is an illustration of a blood flow tube in a stacked folded helix configuration, according to aspects of this disclosure.

FIG. 7A is a top view of an oxygenator system, according to aspects of this disclosure.

FIG. 7B is a side view of an oxygenator system, according to aspects of this disclosure.

FIG. 8A is a side view of an oxygenator system, according to aspects of this disclosure.

FIG. 8B is a top view of an oxygenator system, according to aspects of this disclosure.

FIG. 9 is an illustration of an oxygenator system in a stacked configuration, according to aspects of this disclosure.

FIG. 10 is an illustration of an oxygenator system in a stacked configuration with internal views, according to aspects of this disclosure.

### DETAILED DESCRIPTION OF EMBODIMENTS

Both the foregoing general description and the following detailed description are exemplary and explanatory only and are not restrictive of the features, as claimed. As used herein, the terms “comprises,” “comprising,” “has,” “having,” “includes,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, unless stated otherwise, relative terms, such as, for example, “about,” “substantially,” and “approximately” are used to indicate a possible variation of ±10% in the stated value. In this disclosure, unless stated otherwise, any numeric value may include a possible variation of ±10% in the stated value.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section.

Various embodiments of the present disclosure relate generally to systems for an oxygenator. An oxygenator is a medical device that performs a similar function as a lung, and is designed to expose blood to oxygen, and, in some oxygenators, remove carbon dioxide from the blood. Some oxygenators include a membrane permeable to gas but impermeable to blood. Blood flows on the outside surface of the membrane, while oxygen and medical air flows inside the membrane, which permits the blood cells to absorb oxygen molecules directly. However, some oxygenators impart mechanical fluid forces to the blood, which may lead to adverse outcomes, such as thrombosis and bleeding. Some oxygenators have designs that reduce efficiency of blood flow and oxygen exchange, or have reduced thermal efficiency.

The mechanical fluid forces caused by the shear flow of blood may affect platelet function in vivo, and by determining the link between platelet function and shear stress, one can better comprehend the various biophysical mechanisms governing thrombosis under conditions of blood flow. Thrombosis and bleeding are devastating adverse events in patients supported with blood contacting medical devices. High non-physiological shear stress may cause platelet dysfunction that may contribute to both thrombosis and bleeding.

One or more embodiments may provide an oxygenator with a flexible and modular gas exchange surface area to increase gas exchange surface area with the modular design as clinically indicated. One or more embodiments may provide an oxygenator with a reduced need for heat exchange, based on combination with a mobile pump. One or more embodiments may provide an oxygenator that uses a heat exchange area for oxygen exchange, thus making the same size pump more efficient. One or more embodiments may provide an oxygenator with a low priming circuit that is easy to use given the design of the filling process. One or more embodiments may provide an oxygenator with a sensor for clot burden detection and impact on surface area exposed for exchange. One or more embodiments may provide an oxygenator with a compact design with a display console on the oxygenator and battery pack attached or in line with ports and no need for heat exchange. One or more embodiments may provide an oxygenator with a compact size to be worn on a vest.

One or more embodiments may provide an oxygenator with one or more of (i) an improved inflow and/or outflow design, (ii) a modular design, (iii) an improved membrane, or (iv) an improved flow and/or priming. One or more embodiments may provide a membrane of fiber bundles that is optimized for efficient and laminar blood flow to increase oxygen exchange and reduce mechanical stress on the blood with an optimally shaped canister to encourage the flow. One or more embodiments may provide a system that is designed for optimal laminar flow of blood and also to optimize initial priming of the oxygenator to reduce and remove air in the system before the system engages with the full circulation. One or more embodiments may provide an oxygenator with one or more of (i) reduced shear, (ii) operational and/or volume selection, (iii) improved membrane efficiency, or (iv) reduced pressure loss across the oxygenator inflow and outflow, relative to some designs.

One or more embodiments may provide an oxygenator with an inlet and outlet including a curved design to reduce blood shear and pressure drop. For example, the lumen of each of the inlet tube and/or the outlet tube may have a central axis that curves, and may be defined by a tube wall that is curved. The curved design may include various ranges for an angle of entry. The curved design may include different positions or geometries in a cylinder. The curved design may be helical, for example. One or more embodiments may provide an oxygenator with an inlet and outlet including a funnel outflow to reduce pressure drop or resistance within the system. The funnel outflow may include various ranges for funnel angles and for funnel lengths. The funnel outflow may include different positions in a cylinder. The funnel outflow may include a cylindrical (i.e., constant diameter) outflow downstream from an upstream funnel portion.

There are many advantages to coiled tubes, such as the stabilization effects of turbulent flow and the higher Reynolds number at which the transition from a laminar state to a turbulent state occurs, as compared to straight pipes/tubes. Coiled tubes may reduce the drag reduction from approximately 10% to approximately 30%, as compared to straight pipes. Thus, a coiled tube as all or part of an inlet and/or an outlet of an oxygenator can have flow benefits as compared to straight tubes or tubes with sharp (e.g., 90 degree) bends/corners.

Sharp corners may be areas of high pressure drop and flow reduction with energy loss. One or more embodiments may provide an oxygenator with improved curvature for the flow and pressure requirements of the system and structures such as vanes, deflectors, or straighteners in the areas of highest turbulence. The magnitude of the pressure drop within a curved tube depends on various factors, such as the geometry of the curve (radius of curvature, length of curve, inner diameter of tube, etc.), flow rate, fluid viscosity, and Reynolds number. Corner vanes and deflectors may reduce both turbulence and pressure drop. One or more embodiments may provide an oxygenator with improved tube inner diameter and length.

A reduced pressure drop may improve pump function and efficiency, which may provide a lower shear stress on blood flow. One or more embodiments may provide an oxygenator with a reduced pressure drop at the inflow and/or the outflow based on a modified flow using one or more of vanes, curvatures, deflectors, straighteners, funnels, or dissipation plates. One or more embodiments may provide an oxygenator with a modified cannula inflow diameter with variations for small and large body areas and flow requirements for size or condition.

One or more embodiments may include a blood inflow tube at a lower portion of a canister to deliver blood to the lower portion of the canister. The blood inflow tube may be a coiled or helical inflow, and may deliver blood into the canister to a dissipating surface in the canister. The dissipating surface may be formed with the canister or may be a separate component. The dissipating surface may provide a smooth flow path from the blood inflow tube to a helical hollow fiber membrane. The dissipating surface may include, for example, a separator including optimally angled blades.

One or more embodiments may include a helical hollow fiber membrane in the canister on an opposite side of the dissipating surface from the blood inflow tube. One or more embodiments may include a collecting surface in the canister on an opposite side of the helical hollow fiber membrane from the dissipating surface. The collecting surface may be formed with the canister or may be a separate component. The collecting surface may provide a smooth flow path from the helical hollow fiber membrane to a blood outflow tube. One or more embodiments may include a blood outflow tube at an upper portion of a canister to deliver blood from the upper portion of the canister. The blood outflow tube may be a coiled or helical outflow, and may deliver blood from the dissipating surface and out of the canister. One or more embodiments may include the blood inflow tube at a lower portion of a canister and the blood outflow tube at an upper portion of a canister to reduce bubbles in the blood during a filling, or priming, operation of the oxygenator. One or more of the blood inflow tube or the blood outflow tube may include one or more valves for optimal one way flow and optimal priming to reduce bubbles. These valves may be engaged and disengaged at any time for optimal preparation and operation of the oxygenator.

A blood inflow tube and/or a blood outflow tube may be a standard diameter for connection, such as a ⅜ inch or ½ inch size, for example. A secondary flow (i.e., a cross-sectional circulatory motion) in helical pipes may be caused by centrifugal forces due to the curvature. The helical tube may be a consistent diameter or may be a progressively tapering diameter that is optimal to the curvature of the canister and to optimize laminar flow for the designed pressures and flows for clinical use. Helical pipes may provide stabilization effects of turbulent flow and a higher Reynolds number at which the transition from a laminar state to a turbulent state occurs, compared to straight pipes. One or more of the blood inflow tube or the blood outflow tube may be helical over a portion of a tube (e.g., 10 degrees of the cylinder) or an entirety of a tube (from an entry of the tube to an exit of the tube). One or more of the blood inflow tube or the blood outflow tube may be helical along one or more turns (e.g., 360 degrees of the cylinder or 720 degrees of the cylinder).

Optimizing pitch and creating flow straighteners into the curvature and out of the curvature of the inflow tube and/or the outflow tube may impact reduction of turbulence and reduction of pressure drop. A collecting surface may allow dissipation of shear and evenly gated blood flow distribution. A collecting surface may include a separator. Optimal pitch may be created for blood viscosity in the range from approximately 1 L to approximately 6 L of flow in the appropriate cannula range for engagement with the blood circuit with optimal orientation of the curve pitch and coil winding intensity and optimal consistent or tapering inner diameter of the tube.

One or more embodiments may provide an oxygenator as one or more stackable modules. The stackable modules may allow for volume selection, such that more modules provide a higher volume. The stackable modules may be stackable in a side by side manner or in a top and bottom manner, based on optimal ergonomics and flow paths.

One or more embodiments may provide an oxygenator with a modular/variable volume and with a variable type of oxygenator, for efficiency and applicability to clinical use which may change over the course of an illness. One or more embodiments may provide an oxygenator with one or more modules (e.g., one, two, three, four, or more modules). One or more embodiments may provide an oxygenator with one or more stackable modules. The modules may have the same or different oxygenation capabilities. Any number of modules may be stacked. However, priming the modules or resistance associated with additional modules may provide a practical limit on a number of modules that may be stacked.

One or more embodiments may provide an oxygenator system with stackable modules, where the oxygenator system is controlled the same regardless of a number of stacked modules. One or more embodiments may provide an oxygenator system with stackable modules, where the oxygenator system is controlled differently for different configurations of stacked modules.

The modules may have different functions. For example, a first module may only provide oxygen transfer only, and a second module may provide oxygen transfer and carbon dioxide removal. One or more embodiments may provide modules with a ratio of oxygen transfer volume to carbon dioxide removal volume selected based on a particular application. For example, a ratio may be related to a flow rate, and modules may be selected based on different flow rates and carbon dioxide monitoring. For example, one or more embodiments may provide a module with a higher flow rate to remove carbon dioxide and a lower flow rate to increase oxygen transfer.

One or more embodiments may provide an oxygenator with one or more stackable modules. The modules may include a flow connector or connection design. For example, a flow connector may include a snap connector for medical air. A flow connector may include a spigot connector for blood. A spigot connector may operate with a priming process to fill a module before the module is fully connected.

One or more embodiments may provide an oxygenator system with stackable modules with shared medical air and with separate blood inflow, with a Y-connector to one or more of an inflow cannula and an outflow cannula. For example, an oxygen source may be fluidly and mechanically coupled to each module individually, via a Y-connector. The inflow of the Y-connector connects to the oxygen source, and the outflow branches connect to the oxygen inflow of a corresponding module. The air outflow of each module can connect to an air receiver in a similar way, with a Y-connector.

The blood inflow of a first module may receive blood from a cannula connected to a patient. The blood outflow of that module may connect to a blood inflow of a second module, and the blood outflow of the second module may connect to a cannula for blood return to the patient. Each blood inflow and/or blood outflow may have an on-off valve/spigot in its path to control priming of each module and preventing leaking of blood.

One or more embodiments may provide an oxygenator system with stackable modules for one or more of wearability, transport, or portability. One or more embodiments may provide an oxygenator system with one or more connectors to connect stackable (and/or side-by-side) modules. The one or more connectors may be formed with a canister of the oxygenator system, or may be separate components from the canister. The one or more connectors may include mechanical connections to mechanically connect the modules. Such mechanical connections can include snap connectors, hook-and-loop connectors, or any other suitable connector for conveniently and securely attaching modules in a side-by-side or stacking arrangement.

The one or more connectors may also connect the air/blood inlets and outlets of the modules. Such connectors may have a mechanical connection portion for achieving the fluid connection and a sealing portion for preventing leakage during set-up and/or priming. Such connectors may provide one or more of an air connection between modules, a blood priming operation, or a blood connection between modules. Such connectors may include any medical tubing connectors and valves. The various connectors may provide a stable and leak-free connection between stackable or side-by-side modules.

One or more embodiments may provide an oxygenator with an improved membrane. A membrane may include a fiber bundle construct, for example, based on an optimal winding capability. One or more embodiments may provide a method for producing a membrane. One or more embodiments may provide a membrane design including a helical or double helix design of fibers that is then wound into balls or beads. The balls or beads then may be packed into a sheet, the sheet then may be wound (i.e., similar to rolling up a carpet) and packed into an oxygenator cylinder/canister. One or more embodiments may provide an oxygenator with a denser packing of a membrane for a more compact design. One or more embodiments may provide an oxygenator with a greater surface area of membrane exposed to blood. One or more embodiments may provide an oxygenator with an improved efficiency of oxygen transfer. One or more embodiments may provide an oxygenator that may be worn on a body of a patient. One or more embodiments may provide an oxygenator with no fluid warming required. One or more embodiments may provide an oxygenator with a fluid warmer. A fluid warmer may include a heating coil, for example.

One or more embodiments may provide an oxygenator with a membrane having improved density, compactness, and efficiency. One or more embodiments may provide an oxygenator with a membrane having improved winding and packing. One or more embodiments may provide an oxygenator with one or more spacers within the membrane, to improve flow or insert bioactive materials to prevent adverse events or eluting pharmaceuticals as required. The spacers may be added to the sheet during manufacture of the sheet, for example. The spacers may have an active mechanical function (e.g., agitation triggered by energy such as a magnetic force). The spacers may have an active biologic function (e.g., coating, or eluting pharmaceuticals).

One or more embodiments may provide an oxygenator with a membrane having stacking and shaping for lower pressure drop, higher flow, lower resistance, and higher oxygen transfer efficiency. One or more embodiments may provide an oxygenator with a blood priming flow path to reduce bubbles. One or more embodiments may provide an oxygenator with fast priming, reduced bubbles, uniflow gravity, and low pressure. One or more embodiments may provide an oxygenator with single flow pathways with valves that can be engaged or disengaged as required. One or more embodiments may provide an oxygenator with an improved blood priming flow path using packing and flow design, and a blood priming flow path that may be maintained across a modularity of stackable elements.

One or more embodiments may provide a membrane for an oxygenator. The membrane includes numerous hollow fibers of sizes and materials typically used in oxygenator membranes. The hollow fibers may be wound in a helical shape, including double helix shapes. Loops of the winding may be in a same plane, or the planes of the loops may differ among loops or occur in bundles. The helically wound fibers then may be folded into one or more sheets. The one or more sheets may be rolled or folded, and then placed into a canister of the oxygenator. The hollow fibers may be configured to exchange a gas (such as oxygen) inside the one or more hollow fibers with a fluid (such as blood) outside the hollow fibers.

One or more embodiments may include a membrane to provide a smooth laminar flow and reduced stagnant areas. Membrane boundary layers may improve the laminar flow using one or more of helical stacking or container design for sheet alignment. For example, the boundary of the canister may be optimized for flow and efficiency with a membrane or bundle arrangement. One or more embodiments may include pillars or layers of helical fiber as a membrane. One or more embodiments may include a membrane with inner helical pillars and an outer helix coiled around the helical pillars. One or more embodiments may improve efficiency and flow for durability, reduced blood damage, and better oxygenation. One or more embodiments may include a membrane with an external wrap.

One or more embodiments may provide an oxygenator with one or more of a blood inflow tube or a blood outflow tube. One or more of the blood inflow tube or the blood outflow tube may include a blood flow modifier inside the tube. The blood flow modifier may include one or more of a vane, a separator, or a straightener inside the tube. One or more embodiments may include a blood flow modifier at one or more of an entry or an exit of one or more of the blood inflow tube or the blood outflow tube.

One or more embodiments may provide an oxygenator with a blood flow modifier. The blood flow modifier may modify a flow of blood to reduce a turbulent flow. The blood flow modifier may modify a flow of blood to reduce a pressure drop. A blood flow modifier may include one or more fins (e.g., three fins) extending to a central axis of a blood inflow tube or a blood outflow tube. A blood flow modifier may include one or more of straight portions or angled/pitched portions, relative to a longitudinal direction of flow of blood in the blood inflow tube or blood outflow tube. A blood flow modifier may optimize flow pressure characteristics of the flow in the circuit, as well as provide directional control. One or more embodiments may provide flow management control using bundle and/or sheet design and efficient directional flow using separators and/or valves, for example.

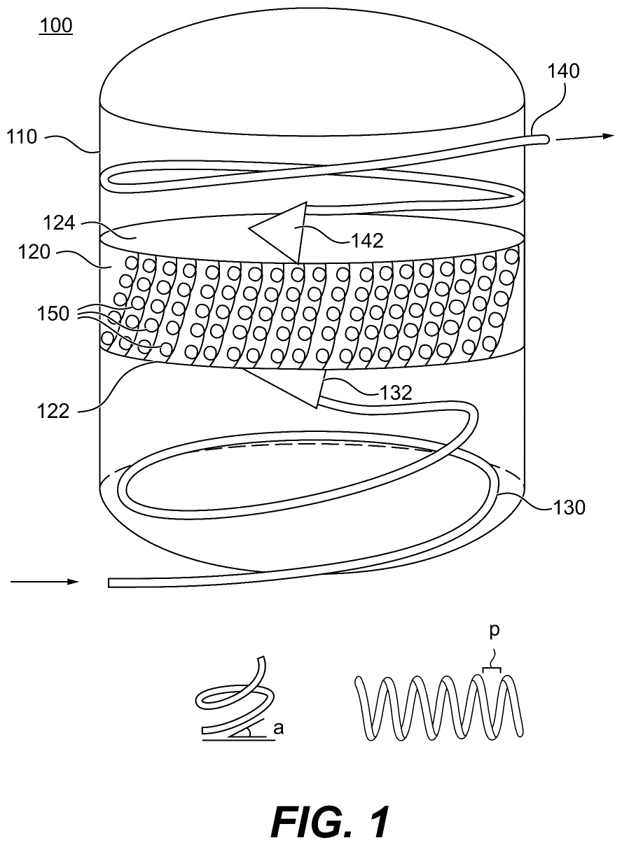

FIG. 1 is an illustration of an oxygenator system, according to aspects of this disclosure. As shown in FIG. 1, the oxygenator system 100 may include a canister 110 having a membrane 120 between a blood inflow tube 130 and a blood outflow tube 140. The blood inflow tube 130 may enter the canister 110 at a lower portion of the canister 110 and deliver blood to the lower portion of the canister 110. The blood outflow tube 140 may enter (or exit) the canister 110 at an upper portion of the canister 110 and deliver blood from the upper portion of the canister 110.

One or more of the blood inflow tube 130 or the blood outflow tube 140 may include a lumen having a curved design or a curved portion to reduce blood shear and pressure drop. For example, the lumen of the blood inflow tube 130 or the blood outflow tube 140 may have a curved design or a curved portion having a central axis that curves, and may be defined by a tube wall that is curved. The curved design or curved portion of the blood inflow tube 130 or the blood outflow tube 140 may include various ranges for an angle “a” of entry (or exit). The angle “a” may be from approximately 5 degrees to approximately 60 degrees, for example, and may be based on one or more of a designed flow or viscosity. The angle “a” may be from approximately 15 degrees to approximately 45 degrees, for example. The curved design or curved portion may include different positions or geometries (i.e., pitch “p”) in the canister 110. The pitch “p” may be a variable pitch as a solution to flow management based on decreasing or increasing pitch to manage flow. The curved portion of the blood inflow tube 130 or the blood outflow tube 140 may form one or more loops within the canister 110. The curved design may reduce blood shear and pressure drop.

For example, the curved design or curved portion of the blood inflow tube 130 or the blood outflow tube 140 may be a coiled or helical portion. The blood inflow tube 130 or the blood outflow tube 140 may be helical over of a section of the blood inflow tube 130 or blood outflow tube 140 within the canister 110 (e.g., approximately 10 degrees of the canister 110) or an entirety of the blood inflow tube 130 or the blood outflow tube 140 within the canister 110 (from an entry of the tube to an exit of the tube). One or more of the blood inflow tube or the blood outflow tube may be helical along one or more turns (e.g., approximately 360 degrees of the canister 110 or approximately 720 degrees of the canister 110).

The blood inflow tube 130 or the blood outflow tube 140 may be a standard diameter for connection, such as approximately ⅜ inch or approximately ½ inch size, for example. The blood inflow tube 130 or the blood outflow tube 140 may be a consistent diameter (e.g., a constant diameter) or may be a progressively tapering diameter that is designed for the curvature of the canister 110 and to optimize laminar flow for the designed pressures and flows for clinical use.

The blood inflow tube 130 may include an inflow funnel portion 132. The blood outflow tube 140 may include an outflow funnel portion 142. The inflow funnel portion 132 and outflow funnel portion 142 may reduce pressure drop or resistance within the canister 110. The inflow funnel portion 132 and outflow funnel portion 142 may include various ranges for funnel angles and for funnel lengths. The inflow funnel portion 132 and outflow funnel portion 142 may include different positions in the canister 110. The outflow funnel portion 142 may include a cylindrical (i.e., constant diameter) outflow downstream from an upstream funnel portion. The inflow funnel portion 132 may include a cylindrical inflow portion upstream from a downstream funnel portion.

The blood inflow tube 130 or the blood outflow tube 140 may include one or more valves (not shown) for optimal one way flow and optimal priming to reduce bubbles. These valves may be engaged and disengaged at any time for optimal preparation and operation of the oxygenator system 100.

The oxygenator system 100 may include a dissipating surface 122 that receives blood from the blood inflow tube 130, and provides a smooth flow path from the blood inflow tube 130 to the membrane 120. The dissipating surface 122 may be formed with the canister 110 or may be a separate component. The dissipating surface 122 may include, for example, a separator including optimally angled blades. The vanes may modify a flow of the blood. The angle may be from approximately 5 degrees to approximately 60 degrees, for example, and may be based on one or more of a designed flow or viscosity. The angle may be from approximately 15 degrees to approximately 45 degrees, for example. The angle may be vertical insertions. The dissipating surface 122 may include, for example, a surface treatment. The dissipating surface 122 may be energized to further transfer of gas. The membrane 120 may be located within the canister 110 on an opposite side of the dissipating surface 122 from the blood inflow tube 130.

The oxygenator system 100 may further include a collecting surface 124 in the canister 110 on an opposite side of the membrane 120 from the dissipating surface 122. The collecting surface 124 may be formed with the canister 110 or may be a separate component. The collecting surface 124 may provide a smooth flow path from the membrane 120 to the blood outflow tube 140. The blood outflow tube 140 may deliver blood from the collecting surface 124 and out of the canister 110.

Optimizing pitch and creating flow straighteners into the curvature and out of the curvature of the inflow tube and/or the outflow tube may impact reduction of turbulence and reduction of pressure drop. Collecting surface 124 may allow dissipation of shear and evenly gated blood flow distribution. Collecting surface 124 may include a separator. Optimal pitch may be created for blood viscosity in the range from approximately 1 L to approximately 6 L of flow in the appropriate cannula range for engagement with the blood circuit with optimal orientation of the curve pitch and coil winding intensity and optimal consistent or tapering inner diameter of the tube.

The membrane 120 may include one or more hollow fibers 150 for exchanging gas with blood. The one or more hollow fibers 150 may be configured to exchange a gas (such as oxygen) inside the one or more hollow fibers 150 with a fluid (such as blood) outside the one or more hollow fibers 150. The one or more hollow fibers 150 may have one or more shapes and may be arranged in one or more directions. The outer diameter of the one or more hollow fibers 150 may be from approximately 300 microns to approximately 400 microns. The bore diameter (inner diameter) of the one or more hollow fibers 150 may be from approximately 200 microns to approximately 280 microns. The wall thickness of the one or more hollow fibers 150 may be from approximately 25 microns to approximately 50 microns.

There may be advantages to blood inflow tube 130 or the blood outflow tube 140 provided as a coiled tube, such as the stabilization effects of turbulent flow and the higher Reynolds number at which the transition from a laminar state to a turbulent state occurs, as compared to straight pipes/tubes. Blood inflow tube 130 or blood outflow tube 140 provided as a coiled tube may reduce the drag reduction from approximately 10% to approximately 30%, as compared to straight pipes. Thus, blood inflow tube 130 or the blood outflow tube 140 of oxygenator system 100 may have flow benefits as compared to straight tubes or tubes with sharp (e.g., approximately 90 degree) bends/corners.

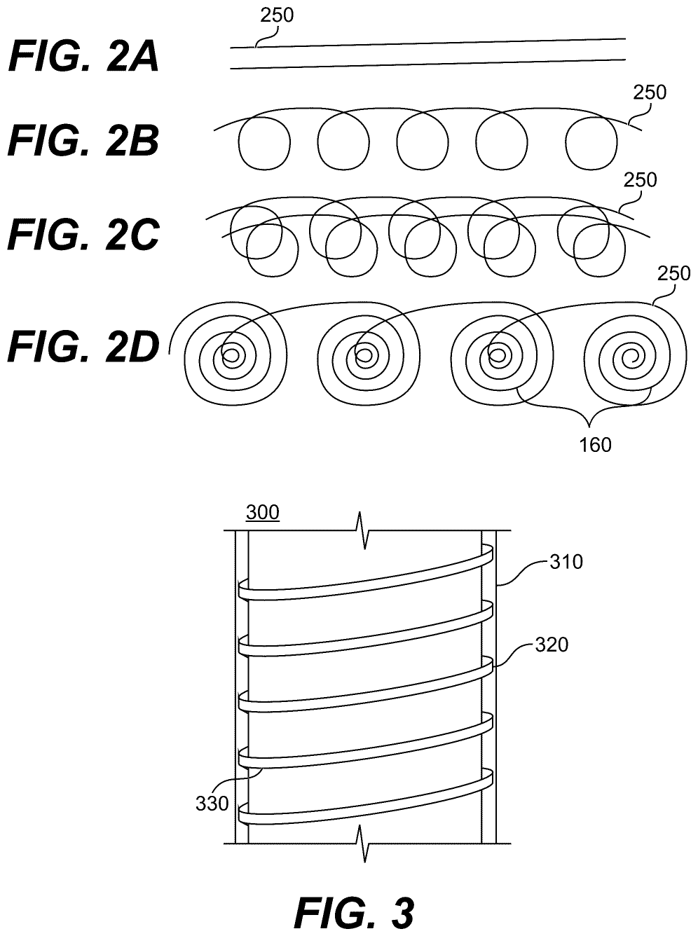

FIG. 2A is an illustration of a hollow fiber for a membrane for an oxygenator system, according to aspects of this disclosure. FIG. 2B is an illustration of the hollow fiber of FIG. 2A formed as a helical winding, according to aspects of this disclosure. FIG. 2C is an illustration of multiple hollow fibers formed as multiple helical windings, according to aspects of this disclosure. FIG. 2D is an illustration of multiple hollow fibers that are folded, according to aspects of this disclosure.

As shown in FIG. 2A, the hollow fibers 250 may include a straight hollow fiber. As shown in FIG. 2B, the hollow fibers 250 may include a helical shape. As shown in FIG. 2C, the hollow fibers 250 may include a double helix shapes, in which two helically shaped hollow fibers 250 have a shared axis about which each of the hollow fibers 250 turns. As shown in FIG. 2C, the hollow fibers 250 may include loops of winding. The loops of winding may be in a same plane, or the planes of the loops may differ among loops or occur in bundles. The hollow fibers 250 may be, for example, helical, or may be folded into one or more sheets. The one or more sheets may be rolled or folded, and then placed into a canister 110 of the oxygenator system 100. As shown in FIG. 2D, the hollow fibers 250 may include one or more spacers 160.

FIG. 3 is an illustration of a membrane of an oxygenator system, according to aspects of this disclosure. FIG. 3 is an illustration of an oxygenator system 300 with a membrane or bundle arrangement 320 having layers of helical fibers which may provide a smooth laminar flow and reduce stagnant areas. The membrane or bundle arrangement 320 may form boundary layers 330 between the membrane or bundle arrangement 320 and a canister 310 using one or more of helical stacking or container design for sheet alignment. The boundary layers 330 may be virtual layers or physical layers, for example. For example, the boundary of the canister 310 may be optimized for flow and efficiency with the membrane or bundle arrangement 320.

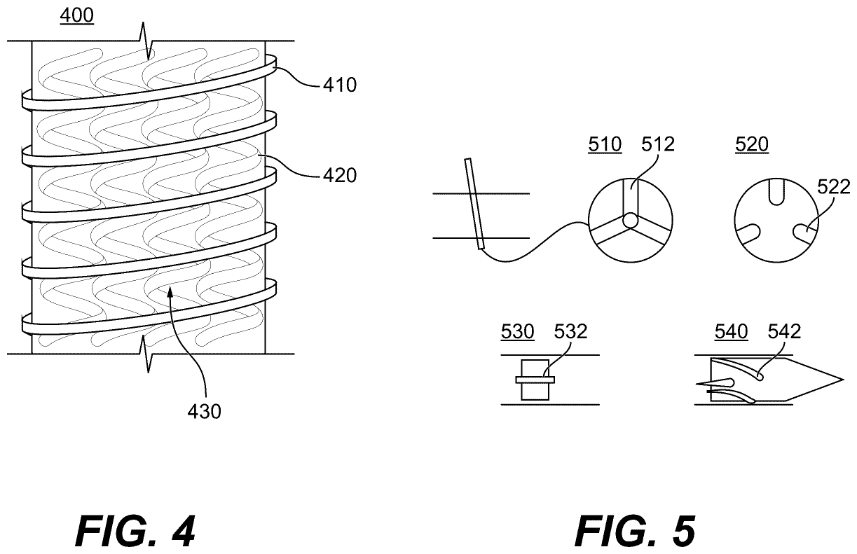

FIG. 4 is an illustration of a membrane with inner and outer fibers, according to aspects of this disclosure. FIG. 4 is an illustration of a membrane 400, including helical fiber pillars 420 and an outer helix 410 coiled around the helical fiber pillars 420. The membrane 400 may further include a blood flow surface 430 optimized for smooth laminar flow. The blood flow surface 430 may include, for example, a surface treatment. Membrane 400 may provide flow management control using bundle and/or sheet design and efficient directional flow using separators and/or valves, for example.

Oxygenator system 100 may include membrane 400 with improvements over some systems. Membrane 400 may include a fiber bundle construct, for example, based on an optimal winding capability. Membrane 400 may include a helical or double helix design of fibers that is then wound into balls or beads. The balls or beads then may be packed into a sheet, the sheet then may be wound (i.e., similar to rolling up a carpet) and packed into an oxygenator cylinder/canister. Oxygenator system 100 may include a denser packing of membrane 400 for a more compact design. Oxygenator system 100 may include a greater surface area of membrane 400 exposed to blood. Oxygenator system 100 may include an improved efficiency of oxygen transfer. Oxygenator system 100 may be worn on a body of a patient. Oxygenator system 100 may not require fluid warming. Oxygenator system 100 may include a fluid warmer. A fluid warmer may include a heating coil, for example.

Oxygenator system 100 may include membrane 400 having improved density, compactness, and efficiency. Oxygenator system 100 may include membrane 400 having improved winding and packing. Oxygenator system 100 may include one or more spacers within membrane 400, to improve flow or insert bioactive materials to prevent adverse events or eluting pharmaceuticals as required. The spacers may be added to the sheet during manufacture of the sheet, for example. The spacers may have an active mechanical function (e.g., agitation triggered by energy such as a magnetic force). The spacers may have an active biologic function (e.g., coating, or eluting pharmaceuticals). Heat dissipation may also be reduced by using phase change materials in the spacers within the oxygenator system 100 or any surface where blood is contained, either directly or indirectly in the canister itself. Thus, the spacers may have an additional purpose being a heat loss and homeothermic heat management function.

Oxygenator system 100 may include membrane 400 having stacking and shaping for lower pressure drop, higher flow, lower resistance, and higher oxygen transfer efficiency. Oxygenator system 100 may include a blood priming flow path to reduce bubbles. Oxygenator system 100 may include fast priming, reduced bubbles, uniflow gravity, and low pressure. Oxygenator system 100 may include single flow pathways with valves that can be engaged or disengaged as required. Oxygenator system 100 may include an improved blood priming flow path using packing and flow design, and a blood priming flow path that may be maintained across a modularity of stackable elements.

Membrane 400 may include numerous hollow fibers of various sizes and materials. The hollow fibers may be wound in a helical shape, including double helix shapes. Loops of the winding may be in a same plane, or the planes of the loops may differ among loops or occur in bundles. The helically wound fibers then may be folded into one or more sheets. The one or more sheets may be rolled or folded, and then placed into a canister of oxygenator system 100. The hollow fibers may be configured to exchange a gas (such as oxygen) inside the one or more hollow fibers with a fluid (such as blood) outside the hollow fibers.

Oxygenator system 100 may include membrane 400 to provide a smooth laminar flow and reduced stagnant areas. Membrane boundary layers may improve the laminar flow using one or more of helical stacking or container design for sheet alignment. For example, the boundary of the canister may be optimized for flow and efficiency with a membrane or bundle arrangement. Oxygenator system 100 may include pillars or layers of helical fiber as membrane 400. Oxygenator system 100 may include membrane 400 with inner helical pillars and an outer helix coiled around the helical pillars. Oxygenator system 100 with membrane 400 may improve efficiency and flow for durability, reduced blood damage, and better oxygenation. Oxygenator system 100 may include membrane 400 with an external wrap.

FIG. 5 is an illustration of blood flow modifiers, according to aspects of this disclosure. A first blood flow modifier 510 may include one or more fins 512 (e.g., three fins) extending to a central axis of a blood inflow tube 130 or a blood outflow tube 140. A central structure may be formed at the conjunction of the one or more fins 512 at or along the central axis. A second blood flow modifier 520 may include one or more fins 522 (e.g., three fins) extending to a central axis of a blood inflow tube 130 or a blood outflow tube 140, with the one or more fins 522 being disjointed. A third blood flow modifier 530 may include a straight portion 532 relative to a longitudinal direction of flow of blood in the blood inflow tube 130 or blood outflow tube 140. A fourth blood flow modifier 540 may include a pitched portion 542 relative to a longitudinal direction of flow of blood in the blood inflow tube 130 or blood outflow tube 140.

In some examples, the blood inflow tube 130 or the blood outflow tube 140 may include first blood flow modifier 510 inside the tube. The first blood flow modifier 510 may include one or more of a vane, a separator, or a straightener inside the tube. First blood flow modifier 510 may be located at one or more of an entry or an exit of one or more of the blood inflow tube 130 or the blood outflow tube 140. First blood flow modifier 510 may modify a flow of blood to reduce a turbulent flow or to reduce a pressure drop. First blood flow modifier 510 may optimize flow pressure characteristics of the flow in the circuit, as well as provide directional control. Second blood flow modifier 520 may be similar to first blood flow modifier 510, or may be different from first blood flow modifier 510.

Oxygenator system 100 may provide a reduced pressure drop to improve function and efficiency, which may provide a lower shear stress on blood flow. Oxygenator system 100 may have a reduced pressure drop at the inflow and/or the outflow based on a modified flow using one or more of vanes, curvatures, deflectors, straighteners, funnels, or dissipation plates. Oxygenator system 100 may include a modified cannula inflow diameter with variations for small and large body areas and flow requirements for size or condition.

Sharp corners may be areas of high pressure drop and flow reduction with energy loss. Oxygenator system 100 may have improved curvature for the flow and pressure requirements of the system and structures such as vanes, deflectors, or straighteners in the areas of highest turbulence. The magnitude of the pressure drop within a curved tube depends on various factors, such as the geometry of the curve (radius of curvature, length of curve, inner diameter of tube, etc.), flow rate, fluid viscosity, and Reynolds number. Corner vanes and deflectors may reduce both turbulence and pressure drop. Oxygenator system 100 may have improved tube inner diameter and length.

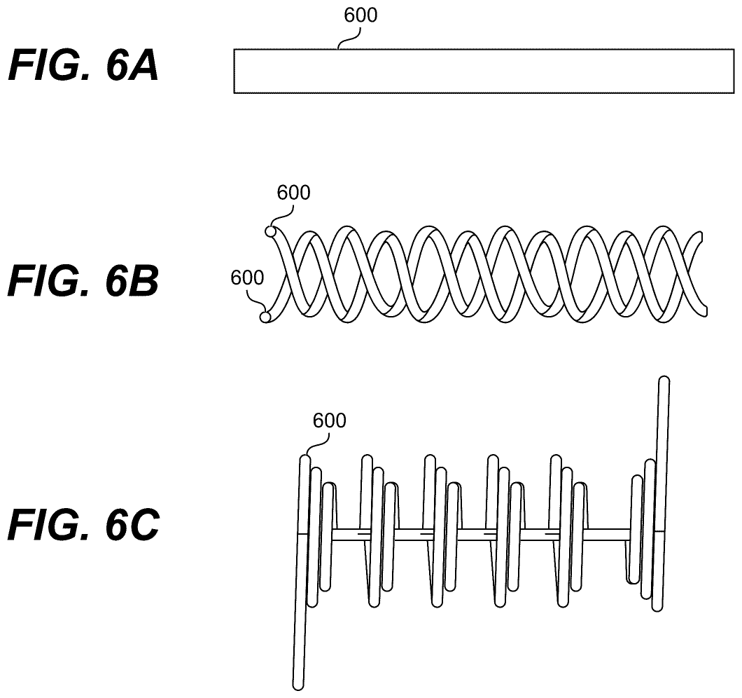

FIG. 6A is an illustration of a straight blood flow tube, according to aspects of this disclosure. FIG. 6B is an illustration of blood flow tubes in a stacked helix configuration, according to aspects of this disclosure. FIG. 6C is an illustration of a blood flow tube in a stacked folded helix configuration, according to aspects of this disclosure. Straight tube 600 may be formed into a stacked helix and folded, as shown in FIG. 6A, FIG. 6B, and FIG. 6C.

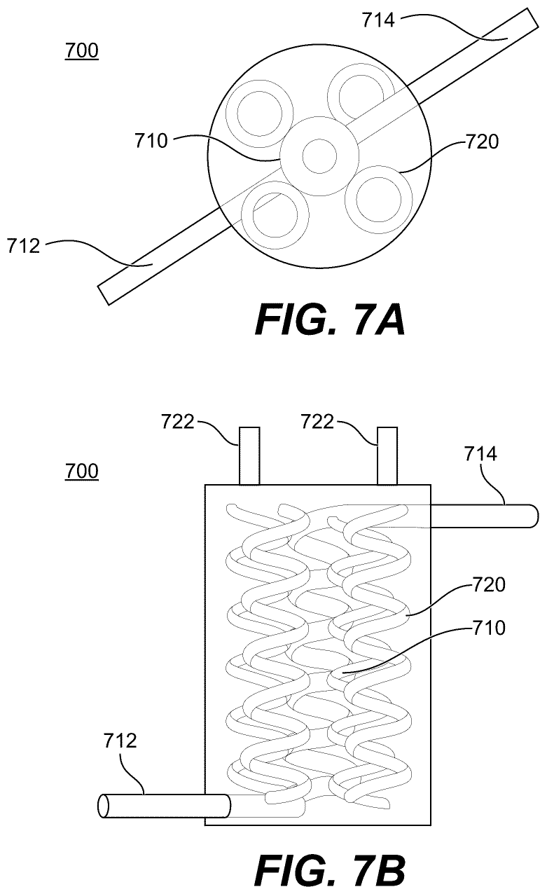

FIG. 7A is a top view of an oxygenator system, according to aspects of this disclosure. FIG. 7B is a side view of an oxygenator system, according to aspects of this disclosure. The oxygenator system 700 may include a blood flow tube 710 in a helical configuration. The blood flow tube 710 may include a blood flow inlet 712 at a bottom portion of the oxygenator system 700 for connection the oxygenator system 700 to a blood source. The oxygenator system 700 may include a blood flow outlet 714 at a top portion of the oxygenator system 700. The oxygenator system 700 may further include one or more blood oxygenation paths 720 that may be enabled for additional oxygenation. The one or more blood oxygenation paths 720 may include inlets (or outlets) 722 for connecting an oxygen supply or destination.

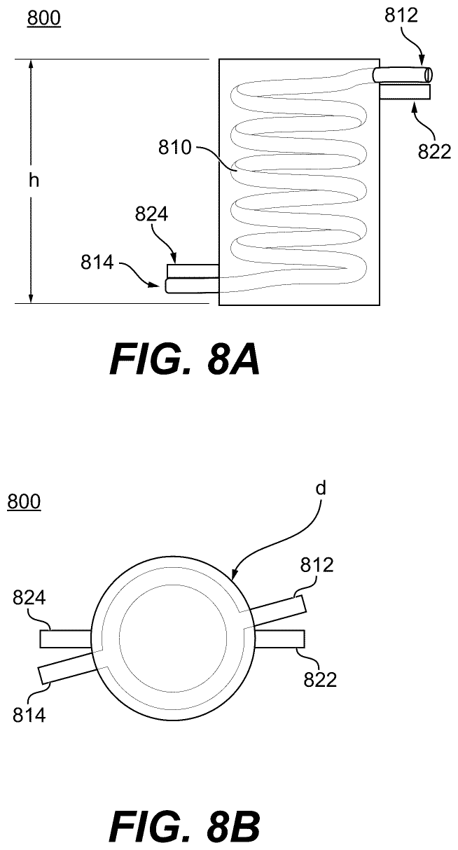

FIG. 8A is a side view of an oxygenator system, according to aspects of this disclosure. FIG. 8B is a top view of an oxygenator system, according to aspects of this disclosure. The oxygenator system 800 may include a blood flow tube 810. Blood flow tube 810 may be helical. The blood flow tube 810 may include a blood flow inlet 812 at a top portion of the oxygenator system 800 for connecting the blood flow tube 810 to a blood source. The oxygenator system 800 may include a blood flow outlet 814 at a bottom portion of the oxygenator system 800. The oxygenator system 800 may include a gas inlet 822 at a top portion of the oxygenator system 800 and a gas outlet 824 at a bottom portion of the oxygenator system 800. The oxygenator system 800 may have an axial height “h” of approximately 5.91 inches and a diameter “d” of approximately 3.94 inches.

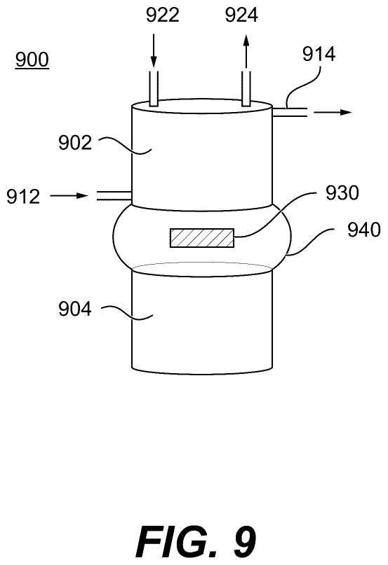

FIG. 9 is an illustration of an oxygenator system in a stacked configuration, according to aspects of this disclosure. The oxygenator system 900 may include a first module 902 connected to a second module 904 by a seal 940 having one or more connectors 930. The one or more connectors 930 may be formed with a canister of the oxygenator system 900, or may be separate components from the canister of the oxygenator system 900. The one or more connectors 930 may include mechanical connections to mechanically connect first module 902 and second module 904. Such mechanical connections can include snap connectors, hook-and-loop connectors, or any other suitable connector for conveniently and securely attaching modules in a side-by-side or stacking arrangement.

The first module 902 may include blood inlet 912, blood outlet 914, gas inlet 922, and gas outlet 924. The blood inlet 912 may be nearer the one or more connectors 930 than the blood outlet 914.

The one or more connectors 930 may also connect the air/blood inlets and outlets of the modules. Such connectors may have a mechanical connection portion for achieving the fluid connection and a sealing portion for preventing leakage during set-up and/or priming. Such connectors may provide one or more of an air connection between modules, a blood priming operation, or a blood connection between modules. Such connectors may include any medical tubing connectors and valves.

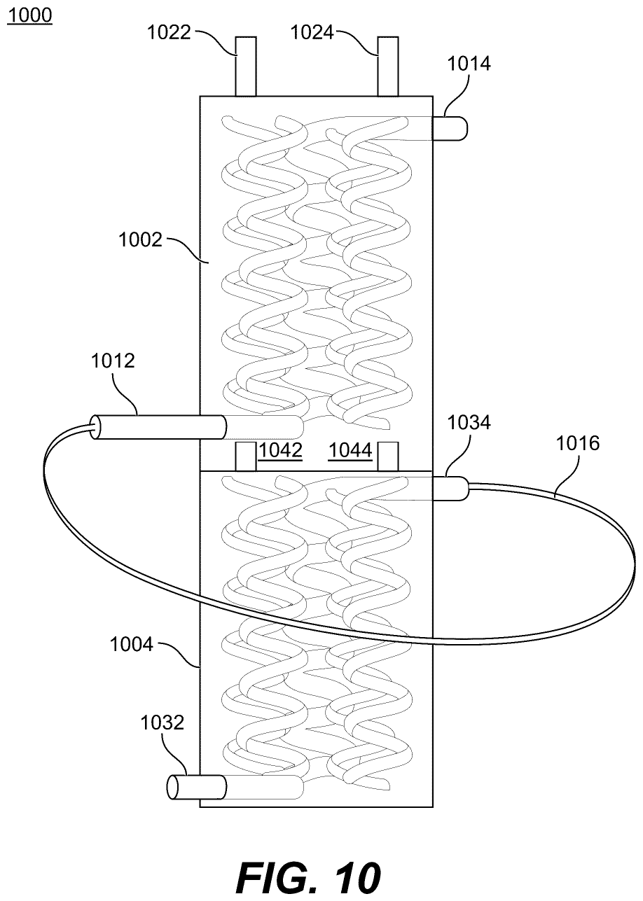

FIG. 10 is an illustration of an oxygenator system in a stacked configuration with internal views, according to aspects of this disclosure. Oxygenator system 1000 may include first canister 1002, second canister 1004, and connector 1016. Each of first canister 1002 and second canister 1004 may be used independently (i.e., using first canister 1002 without second canister 1004 or vice versa, as described with reference to FIG. 1, for example) or may be used together in a stacked system as shown in FIG. 10.

First canister 1002 may include first blood flow inlet 1012, first blood flow outlet 1014, first gas inlet 1022, and first gas outlet 1024. Second canister 1004 may include second blood flow inlet 1032, second blood flow outlet 1034, second gas inlet 1042, and second gas outlet 1044. Connector 1016 may connect second blood flow outlet 1034 to first blood flow inlet 1012. Second gas inlet 1042 and second gas outlet 1044 may mate with corresponding connections in first canister 1002. First canister 1002 have either internal or external connectors that allow flow from one canister to the other without disturbance and is non-reversible. This may expand the capacity of the surface being exchanged, and the modularity of oxygenator system 1000 may allow specificity of exchange and greater surface area. Connector 1016 may snap on and allow blood exchange similarly to connectors in IV circuits, or the connection may be built in and opened with a valve, for example.

Oxygenator system 1000 may include one or more stackable modules (e.g., first canister 1002, second canister 1004). The stackable modules may allow for volume selection, such that more modules provide a higher volume. The stackable modules may be stackable in a side by side manner or in a top and bottom manner, based on optimal ergonomics and flow paths.

The stackable modules may provide a modular/variable volume and with a variable type of oxygenator, for efficiency and applicability to clinical use which may change over the course of an illness. Oxygenator system 1000 may include one or more modules (e.g., one, two, three, four, or more modules). The modules may have the same or different oxygenation capabilities. Any number of modules may be stacked. However, priming the modules or resistance associated with additional modules may provide a practical limit on a number of modules that may be stacked.

Oxygenator system 1000 may be controlled the same regardless of a number of stacked modules. Oxygenator system 1000 may be controlled differently for different configurations of stacked modules.

First canister 1002 and second canister 1004 may have different functions. For example, first canister 1002 may only provide oxygen transfer only, and second canister 1004 may provide oxygen transfer and carbon dioxide removal. Oxygenator system 1000 may include modules with a ratio of oxygen transfer volume to carbon dioxide removal volume selected based on a particular application. For example, a ratio may be related to a flow rate, and modules may be selected based on different flow rates and carbon dioxide monitoring. For example, first canister 1002 may operate with a higher flow rate to remove carbon dioxide and a lower flow rate to increase oxygen transfer.

First canister 1002 and second canister 1004 may include a flow connector or connection design. For example, a flow connector may include a snap connector for medical air. A flow connector may include a spigot connector for blood. A spigot connector may operate with a priming process to fill first canister 1002 before the module is fully connected.

First canister 1002 and second canister 1004 may have shared medical air and with separate blood inflow, with a Y-connector to one or more of an inflow cannula and an outflow cannula. For example, an oxygen source may be fluidly and mechanically coupled to each module individually, via a Y-connector. The inflow of the Y-connector connects to the oxygen source, and the outflow branches connect to the oxygen inflow of a corresponding module. The air outflow of each module can connect to an air receiver in a similar way, with a Y-connector.

The blood inflow of first canister 1002 may receive blood from a cannula connected to a patient. The blood outflow of first canister 1002 may connect to a blood inflow of second canister 1004, and the blood outflow of second canister 1004 may connect to a cannula for blood return to the patient. Each blood inflow and/or blood outflow may have an on-off valve/spigot in its path to control priming of each module and preventing leaking of blood.

Oxygenator system 1000 may include stackable modules (e.g., first canister 1002, second canister 1004) for one or more of wearability, transport, or portability. Oxygenator system 1000 may include one or more connectors to connect stackable (and/or side-by-side) modules. The one or more connectors may be formed with a canister (e.g., first canister 1002, second canister 1004) of the oxygenator system, or may be separate components from the canister. The one or more connectors may include mechanical connections to mechanically connect the modules. Such mechanical connections can include snap connectors, hook-and-loop connectors, or any other suitable connector for conveniently and securely attaching modules in a side-by-side or stacking arrangement.

The one or more connectors may also connect the air/blood inlets and outlets of the modules. Such connectors may have a mechanical connection portion for achieving the fluid connection and a sealing portion for preventing leakage during set-up and/or priming. Such connectors may provide one or more of an air connection between modules, a blood priming operation, or a blood connection between modules. Such connectors may include any medical tubing connectors and valves. The various connectors may provide a stable and leak-free connection between stackable or side-by-side modules.

One or more embodiments may provide an oxygenator with one or more of (i) an improved inflow and/or outflow design, (ii) a modular design, (iii) an improved membrane, or (iv) an improved flow and/or priming. One or more embodiments may provide a membrane of fiber bundles that is optimized for efficient and laminar blood flow to increase oxygen exchange and reduce mechanical stress on the blood with an optimally shaped canister to encourage the flow. One or more embodiments may provide a system that is designed for optimal laminar flow of blood and also to optimize initial priming of the oxygenator to reduce and remove air in the system before the system engages with the full circulation. One or more embodiments may provide an oxygenator with one or more of (i) reduced shear, (ii) operational and/or volume selection, (iii) improved membrane efficiency, or (iv) reduced pressure loss across the oxygenator inflow and outflow, relative to some designs.

One or more embodiments may provide an oxygenator with a flexible and modular gas exchange surface area to increase gas exchange surface area with the modular design as clinically indicated. One or more embodiments may provide an oxygenator with a reduced need for heat exchange, based on combination with a mobile pump. One or more embodiments may provide an oxygenator that uses a heat exchange area for oxygen exchange, thus making the same size pump more efficient. One or more embodiments may provide an oxygenator with a low priming circuit that is easy to use given the design of the filling process. One or more embodiments may provide an oxygenator with a sensor for clot burden detection and impact on surface area exposed for exchange. One or more embodiments may provide an oxygenator with a compact design with a display console on the oxygenator and battery pack attached or in line with ports and no need for heat exchange. One or more embodiments may provide an oxygenator with a compact size to be worn on a vest.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An oxygenator system comprising:
a canister;
a membrane in the canister to provide a gas transfer with blood;
a blood inflow tube to deliver blood to the canister; and
a blood outflow tube to deliver blood from the canister,
wherein one or more of the blood inflow tube or the blood outflow tube includes a curved portion.

2. The oxygenator system of claim 1, wherein one or more of the blood inflow tube or the blood outflow tube includes a blood flow modifier.

3. The oxygenator system of claim 2, wherein the blood flow modifier includes fins extending towards a central axis of the one or more of the blood inflow tube or the blood outflow tube.

4. The oxygenator system of claim 2, wherein the blood flow modifier includes a straight portion relative to a longitudinal direction of flow.

5. The oxygenator system of claim 2, wherein the blood flow modifier includes an angled or pitched portion relative to a longitudinal direction of flow.

6. The oxygenator system of claim 1, wherein the curved portion is helical.

7. The oxygenator system of claim 1, wherein one or more of the blood inflow tube or the blood outflow tube includes a funnel portion.

8. The oxygenator system of claim 1, further comprising:
a dissipating surface between the membrane and one or more of the blood inflow tube or the blood outflow tube.

9. The oxygenator system of claim 8, wherein the dissipating surface further includes a separator including angled blades.

10. The oxygenator system of claim 1, further comprising:
a dissipating surface between the membrane and one or more of the blood inflow tube or the blood outflow tube.

11. An oxygenator system comprising:
a first canister; and
a connector to connect the first canister to a second canister.

12. The oxygenator system of claim 11, wherein:
the first canister includes a first blood flow inlet and a first blood flow outlet, and
the second canister includes a second blood flow inlet to connect to the first blood flow outlet, and a second blood flow outlet.

13. The oxygenator system of claim 11, wherein:
the first canister includes a first gas inlet and a first gas outlet, and
the second canister includes a second gas inlet to connect to the first gas outlet, and a second gas outlet.

14. The oxygenator system of claim 11, wherein the first canister includes a first stacking feature, and the second canister includes a second stacking feature to connect to the first stacking feature to stack the second canister on the first canister.

15. The oxygenator system of claim 11, wherein:
the first canister is configured for a first flow rate,
the second canister is configured for a second flow rate different from the first flow rate, and
the first flow rate and the second flow rate include one or more of a gas flow rate or a blood flow rate.

16. The oxygenator system of claim 11, wherein:
the first canister is configured to transfer a first gas into blood, and
the second canister is configured to remove a second gas from blood.

17. A membrane for an oxygenator system, the membrane comprising:
one or more hollow fibers that are each wound in a helical shape,
wherein the one or more hollow fibers are configured to exchange a gas inside the one or more hollow fibers with a fluid outside the one or more hollow fibers.

18. The membrane of claim 17, wherein the membrane includes one or more spacers to provide an additional function to the membrane.

19. The membrane of claim 17, wherein the one or more hollow fibers include inner helical pillars folded into one or more sheets and an outer helix coiled around the one or more sheets.

20. The membrane of claim 17, wherein the helical shape includes double helix shapes.
